(19) United States
(12) Patent Application Publication
(10) Pub. No.: US 2004/0091520 A1
(43) Pub. Date: May 13, 2004

(54) Disposable gloves with at least one alpha hydroxy acid

(76) Inventor: Samuel Amdur III, Libertyville, IL (US)

Correspondence Address: JENKENS & GILCHRIST, P.C., 225 WEST WASHINGTON, SUITE 2600, CHICAGO, IL, 60606, US

(21) Appl. No.: 10/701,863

(22) Filed: Nov. 5, 2003

Related U.S. Application Data
(60) Provisional application No. 60/425,652, filed on Nov. 12, 2002.

(51) Int. Cl. A61K 35/78; A61K 9/70; B05D 3/00

(52) U.S. Cl. 424/443; 424/744; 427/2.3

(57) ABSTRACT

A disposable examination glove is made by forming a disposable glove from a flexible material; coating the interior surface of the glove with a liquid carrier, Aloe Vera, and at least one alpha hydroxy acid; and removing liquid carrier from the coating to form a substantially dry coating of Aloe Vera and the at least one alpha hydroxy acid on the interior surface of the glove. The dry coating is bonded to the interior surface of the glove so that the coating contacts the hand of a person wearing the glove.

### RELATED APPLICATION

This application claims the benefit of Provisional Application No. 60/425,652 entitled “Disposable Glove with Therapeutic Coating” filed on Nov. 12, 2002, which is hereby incorporated by reference in its entirety.

### FIELD OF THE INVENTION

The present invention relates to disposable protective gloves, such as the latex gloves that are used for examination purposes in the medical field. More particularly, this invention relates to such gloves that have a coating on the inside surface of each glove.

### BACKGROUND OF THE INVENTION

U.S. Pat. Nos. 6,274,154 and 6,423,328 describe disposable gloves that are coated on the inside with dehydrated Aloe Vera. Such gloves have been well received in the marketplace.

Disposable gloves are widely used as a protective measure to protect the person wearing the gloves from various objects or materials handled or touched by that person. To facilitate the handling of objects by the person wearing the gloves, and to maximize the wearer's tactile senses, disposable gloves are made of thin and elastic material that minimize the space between the skin and the glove. Due to poor air circulation within the gloves while they are being worn, hand sweating is a common problem among glove wearers. Prolonged wearing of disposable gloves causes a moist environment on the surface of the hand that allows viruses, bacteria, yeast and fungus to grow and multiply. Itchiness is a frequent result of wearing disposable examination gloves for extended periods.

There is therefore a need for low-cost disposable gloves that can apply moisturizing and therapeutic substances to the hands while the gloves are being worn, while at the same time retaining the desirable characteristics and functions of conventional disposable gloves.

### SUMMARY OF THE INVENTION

In accordance with the present invention, there is provided a method of manufacturing a disposable examination glove comprising

forming a disposable glove from a flexible material,

coating the interior surface of the glove with a liquid carrier, Aloe Vera, and at least one alpha hydroxy acid, and

removing liquid carrier from the coating to form a substantially dry coating of Aloe Vera and the at least one alpha hydroxy acid on the interior surface of the glove, the coating being attached to the interior surface of the glove so that the coating contacts the hand of a person wearing the glove.

The present invention also provides a disposable glove comprising

a flexible material forming a cavity shaped to receive a hand, and

a coating on the interior surface of the cavity for contacting a hand inserted into the cavity, the coating comprising Aloe Vera and at least one alpha hydroxy acid.

In one embodiment of the invention, there is provided a disposable examination glove comprising

a flexible material forming a cavity shaped to receive a hand, and

a therapeutic coating on the interior surface of the cavity for contacting a hand inserted into the cavity, the therapeutic coating consisting essentially of Aloe Vera and at least one alpha hydroxy acid, the coating being formed by applying a liquid carrier containing Aloe Vera and the at least one alpha hydroxy acid to the interior surface, and then heating the coating to evaporate the liquid carrier and form a dehydrated coating of Aloe Vera and the at least one alpha hydroxy acid bonded to said flexible material.

The disposable gloves are generally made of one of three types of materials: natural rubber latex, acrylonitrile, or polyvinyl chloride. Processes for making such gloves, and the specific materials to be used, are well known in this art. Alternative materials for making the disposable gloves are polyurethane, chloroprene, or neoprene.

Aloe Vera is a natural plant extract that has a long history of folk medicine usage. Aloe Vera has been used for external treatment of wounds, bums and skin irritations, and internal treatment of various conditions. Aloe Vera is a popular ingredient in skin-care products. It is also an anti-inflammatory and anti-microbial agent. Aloe Vera is soluble in water and contains non-detectable oil content.

Alpha hydroxy acids (“AHAs”) are naturally occurring, low-molecular-weight, organic acids derived from the sugars in particular plants or milk. Examples are glycolic acid derived from sugar cane, lactic acid derived from milk, tartaric acid derived from grapes, citric acid derived from citrus fruits, malic acid derived from apples, and mandelic acid derived from almonds. AHAs have a carboxylic acid group (—COOH), and a hydroxyl group (—OH) on the carbon atom immediately adjacent to the acid group, i.e., the “alpha” position.

### BRIEF DESCRIPTION OF THE DRAWINGS

The invention may best be understood by reference to the following description taken in conjunction with the accompanying drawings, in which:

FIG. 1 is the front perspective view of a disposable glove embodying the present invention.

FIG. 2 is a sectional view taken along the lines 2-2 of FIG. 1.

### DETAILED DESCRIPTION OF THE PREFERRED EMBODIMENTS

Although the invention will be described next in connection with certain preferred embodiments, it will be understood that the invention is not limited to those particular embodiments. On the contrary, the description of the invention is intended to cover all alternatives, modifications, and equivalent arrangements as may be included within the spirit and scope of the invention as defined by the appended claims.

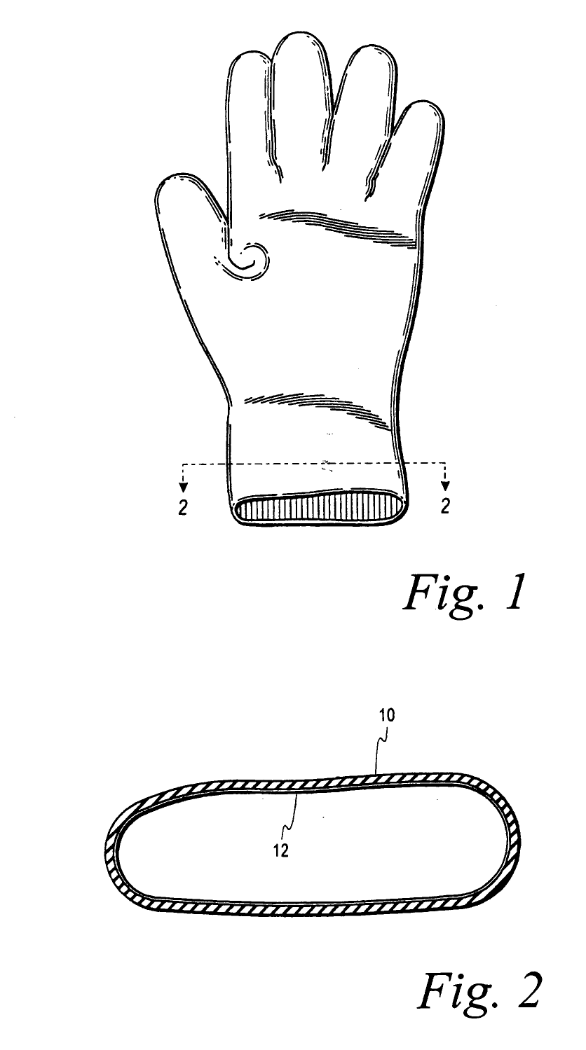

The disposable gloves that are provided with interior coatings according to the present invention are initially formed by conventional glove-manufacturing processes that are well known in the industry. The invention is particularly useful for examination gloves or surgical gloves, which are typically made of a thin material so that the person wearing the gloves still has a good sense of feel through the gloves. As already mentioned, most such gloves are made of a single layer of natural latex rubber, or acrylonitrile, or poylvinyl chloride. One example of such a glove 10 is illustrated in FIGS. 1 and 2 of the drawings.

Techniques for cleaning items such as gloves of surface contaminants, e.g., using a chlorine solution, are well known. Following the cleaning treatment, with the glove turned inside out, the glove is dipped into a liquid carrying the materials to be applied to the glove to form the desired coating 12 on the glove surface that will be the interior surface in normal use. The liquid may be agitated during dipping. When the glove is removed from the liquid, a certain amount of the liquid clings to the glove. The amount of liquid that clings to the glove can be controlled by controlling the viscosity of the liquid. Alternatively, the coating liquid may be applied to the glove by spraying or dripping, or the glove may be dipped into the liquid, after which the glove is dried and stripped from the former, with no off-line processing.

To prepare the liquid into which the gloves are dipped, water is added to an aloe powder concentrate to produce an aloe gel. For example a 200:1 powder concentrate may be mixed with 200 parts water to produce a 100% gel, or 400 parts water to produce a 25% gel. Next, the therapeutic coating, alpha hydroxy acid (AHA), is added to the liquid carrier, preferably at a concentration level that is within the range from about 5% to about 15% by weight. In one specific example, the liquid carrier contains 20% by weight Aloe Vera and 10% by weight AHA.

The liquid that is used to coat the gloves should not contain any ingredients that have a deleterious effect on the strength or other properties of the base material of the glove over the anticipated shelf life of the gloves. For example, natural rubber latex is sensitive to oil-based substances, and thus the liquid used to coat gloves made of natural rubber latex should not contain oil-based substances that have a deleterious effect on such gloves during their anticipated shelf life. A preferred liquid for coating the gloves contains only water, Aloe Vera, and AHA. It is contemplated that other therapeutic coatings, such as allantoin, may be added with the AHA. The preferred liquid carrier is water, and distilled water is most preferred.

After the glove has been coated with the liquid, the glove is dried to remove a sufficient amount of the liquid carrier to form a substantially dry coating bonded to the glove surface. More specifically, the liquid carrier is removed by evaporation, leaving the mixture of Aloe Vera and AHA as a substantially dry coating bonded to the glove surface. The dry coating is preferably distributed substantially uniformly over the glove surface. After the glove has cooled to room temperature, the glove is turned right-side-out so that the coating is on the inside surface of the glove. The solid coating preferably has a thickness of less than about 0.01 millimeter (mm).

After the gloves have been dried, they are allowed to cool to room temperature. The gloves are then inverted so that the coated surface is the interior surface of the gloves.

The coating may be applied by spraying a batch of clean loose gloves that are arranged inside out. The gloves are preferably tumbled while they are being sprayed with a fine mist of the coating liquid so that the spray coats the gloves substantially uniformly. The tumbling of the gloves is preferably continued for a short time after the spraying has stopped. These steps may be repeated two or more times to ensure that the gloves are adequately coated.

For example, for a batch of about 3000 gloves, two kilograms of coating liquid may be sprayed in 4 or 5 spray iterations lasting about 30 to 90 seconds each and spaced 2 to 5 minutes apart. In one embodiment, the spraying is carried out in a dryer in which the maximum temperature is limited to about 80° C., preferably less than about 65° C. Each spray iteration is preferably followed by a tumbling iteration, the last of which is of sufficient duration to complete the drying of the gloves. For example, the final iteration of tumbling may be chosen so that the total duration of tumbling and heating the gloves over all the steps is from about 35 to about 40 minutes.

Preferably, the process is carried out using only two or only three containers in which washing, spraying, and tumbling are performed. If two containers are used, they would typically be a chlorine washer and a heat tumble dryer. If three containers are used, they would typically be a chlorine washer, a water washer, and a heat tumble dryer.

In another process, the coating of the gloves is integrated with, and/or includes, a conventional process for manufacturing the underlying gloves themselves. This modified process is especially preferred for producing large quantities of coated gloves efficiently.

A conventional process for manufacturing examination gloves forms the gloves on molds. Each of the molds has the general shape of a hand so that the resulting gloves will fit hands. The gloves are formed on the molds by a conventional process normally used to produce gloves of the desired material, preferably fully automated within a production line. The gloves that are formed on the mold are considered to be inside out such that the interior of each glove, as later to be worn on the hand, faces outward. After the gloves are formed, and while still on the mold, the outwardly facing surfaces of the gloves are preferably made safer for later contact with hands, and/or easier to slide during donning, either by using chlorine to clean off any residual proteins, chemicals, and the like, or by coating the surface with a thin insulating layer to insulate the hand from contact with the residual proteins, chemicals, and the like. The insulating layer is preferably made of a substance that is more slippery than the underlying glove, such as a polymer layer of silicone or polyurethane.

After the gloves have been formed and cleaned and/or coated with an insulating layer, the coating liquid is applied to the gloves while the gloves are still on the molds. The application of the coating can be by spraying, immersing, pouring, dripping, dipping (which are not mutually exclusive techniques), or any other suitable technique. If spraying is used to apply the coating liquid to the gloves, the spraying should be thorough enough so as to leave a desired amount of solution on the outwardly facing surfaces of the gloves, e.g., an amount comparable to that obtained by dipping.

The liquid coating is then at least partially dried on the gloves, while they are still on the molds, such as by blowing heated air across the gloves on the molds or drying them in an oven. For natural rubber latex gloves, the air is preferably less than about 80° C., and most preferably is less than about 65° C. Preferably, the coating is dried sufficiently to provide adhesion between the coating and the glove so that the coated glove can withstand the step of stripping the glove from the mold. After the gloves are removed from the molds, the loose gloves may be further dried and cured by heating.

One example of a known process for forming and processing of gloves on molds on an automatic production line is as follows: cleaning porcelain formers (molds) using hot water (for example, about 40° C. to 100° C.); drying the porcelain formers in hot air (for example, at about 40° C. to 100° C.) dipping the formers in coagulant; drying the coagulant on the formers in hot air (for example, at about 35° C. to 140° C.); dipping the coagulant-coated formers in latex (for example, at about 25° C. to 45° C.); curing the latex on the formers in hot air (for example, at about 60° C. to 140° C.); leaching the gloves on the formers; beading the edges of the gloves on the formers; and then making the glove surfaces safer, and easier to don, either by cleaning or by coating the surface, as discussed above. If cleaning is used, then the forming and processing further includes: further curing (for example, at about 80° C. to 140° C.); rinsing with cold water (for example, at no more than room temperature); chlorination (for example, at no more than about 30° C.), preferably preceded by further rinsing with cold water (for example, at no more than room temperature); neutralization; further rinsing (for example, with hot followed by cold water); and drying and further curing in hot air. Alternatively, if an insulating coating is used, then the forming and processing further includes: drying in hot air (for example, at about 80° C. to 150° C.); coating with polymer (for example, at no more than about 45° C.); and further drying and curing in hot air (for example, at about 80° C. to 150° C.).

While the invention is described in some detail with specific reference to a few preferred embodiments and some alternatives, there is no intent to limit the invention to the particular embodiments or the specific alternatives. Thus, the true scope of the present invention is not limited to any one of the foregoing exemplary embodiments.

1. A method of manufacturing a disposable examination glove comprising:
forming a disposable glove from a flexible material;
coating an interior surface of said glove with a liquid carrier, Aloe Vera, and at least one alpha hydroxy acid, and
removing said liquid carrier from the coating to form a substantially dry coating of Aloe Vera and said at least one alpha hydroxy acid, said coating being attached to said interior surface of said glove so that the coating contacts the hand of a person wearing said glove.

2. The method of claim 1 wherein said disposable glove is made from a natural rubber latex.

3. The method of claim 1 wherein said liquid carrier is removed from said coating by evaporation.

4. The method of claim 1 wherein said glove is made of a single layer of flexible material prior to being coated.

5. The method of claim 1 wherein said liquid carrier is water.

6. The method of claim 1 wherein said coating is formed by dipping said glove into said liquid carrier containing Aloe Vera and said at least one alpha hydroxy acid.

7. The method of claim 1 wherein said coating is formed by spraying said glove with said liquid carrier containing Aloe Vera and said at least one alpha hydroxy acid.

8. The method of claim 1 wherein said glove is inside out when said coating is applied to said interior surface.

9. The method of claim 1 wherein said glove is turned right side out after evaporating said liquid carrier from the coating to form a substantially dry coating attached to said interior surface.

10. A method of manufacturing a disposable examination glove comprising:
forming a disposable glove from a single layer of flexible material;
applying a coating of a liquid carrier, Aloe Vera, and at least one alpha hydroxy acid, to an interior surface of said glove; and
removing said liquid carrier to form a substantially dry coating of Aloe Vera and said at least one alpha hydroxy acid, said coating being attached to said interior surface of said glove so that the coating contacts the hand of a person wearing said glove.

11. A method of manufacturing a disposable examination glove comprising:
forming a disposable glove from a single layer of natural rubber latex;
applying a coating of a liquid carrier, Aloe Vera, and at least one alpha hydroxy acid to the surface of said glove that is normally the inner surface of said glove but is the outer surface when said coating is applied;
removing said liquid carrier to form a substantially dry coating of Aloe Vera and said at least one alpha hydroxy acid, said coating being attached to the surface of said glove while said glove is still turned inside out; and
turning the glove right side out, so that the coating contacts the hand of a person wearing said glove.

12. A method of reducing the adverse effects of a disposable glove on the skin of a person wearing the glove, comprising coating the interior surface of the glove with Aloe Vera and at least one alpha hydroxy acid, said coating being attached to said interior surface.

13. A disposable glove comprising:
a flexible material forming a cavity shaped to receive a hand; and
a coating on the interior surface of said cavity for contacting a hand inserted into said cavity, said coating comprising Aloe Vera and at least one alpha hydroxy acid.

14. The disposable glove of claim 13 wherein said flexible material is selected from the group consisting of natural latex rubber, acrylonitrile, and polyvinyl chloride.

15. The disposable glove of claim 13 wherein said flexible material consists of only a single layer.

16. The disposable glove of claim 13 wherein said coating is distributed substantially evenly over said interior surface.

17. The disposable glove of claim 13 wherein said coating is formed by applying a liquid carrier containing Aloe Vera and at least one alpha hydroxy acid to said interior surface, and then removing liquid carrier from the coating to form a substantially dry coating of Aloe Vera and said at least one alpha hydroxy acid.

18. The disposable glove of claim 13 wherein said disposable glove is an examination glove.

19. The disposable glove of claim 13 wherein said coating consists essentially of Aloe Vera and at least one alpha hydroxy acid.

20. The disposable glove of claim 19 wherein said alpha hydroxy acid is glycolic acid.

21. A disposable examination glove comprising:
a flexible material forming a cavity shaped to receive a hand, said flexible material consisting essentially of at least one material selected from the group consisting of natural latex rubber, acrylonitrile and polyvinyl chloride; and
a coating on the interior surface of said cavity for contacting a hand inserted into said cavity, said coating consisting essentially of Aloe Vera and at least one alpha hydroxy acid, said coating being formed by applying a liquid carrier containing Aloe Vera and said at least one alpha hydroxy acid to said interior surface, and then heating the coating to evaporate liquid carrier and form a substantially dry coating of Aloe Vera and said at least one alpha hydroxy acid bonded to said flexible material.

22. A method of manufacturing protective gloves, the method comprising:
forming a glove on a mold, applying a liquid carrier containing Aloe Vera and at least one alpha hydroxy acid to said glove while the glove is on the mold so as to form a coating, removing said liquid carrier from said coating to form a substantially dry coating of Aloe Vera and said at least one alpha hydroxy acid while the glove is on the mold, and removing the glove from the mold.

23. The method of claim 22 wherein the forming, applying, removing said liquid carrier, and removing said glove are performed on an automatic production line.

24. The method of claim 22 wherein the removing of said liquid carrier includes evaporating liquid carrier from the coating that has been applied onto the glove while the glove is on the mold.

25. The method of claim 22 including further drying the coating that has been applied onto the glove after the glove is no longer on the mold.

26. The method of claim 22 wherein the applying of said liquid carrier comprises spraying or dripping said liquid carrier onto the glove while the glove is on the mold.

27. The method of claim 22 wherein said glove on said mold is considered to be inside out, whereby the outwardly facing surface of said glove that is on said mold is the surface that will face a hand when the glove is worn.

28. The method of claim 22 wherein the applying of said liquid carrier comprises dipping said glove while said glove is on said mold into a quantity of said liquid carrier.

29. The method of claim 28 wherein said glove on said mold is considered to be inside out, whereby the outwardly facing surface of said glove that is on said mold is the surface that will face a hand when said glove is worn.

30. The method of claim 22 wherein said coating applied to said glove consists essentially of water, Aloe Vera, and said at least one alpha hydroxy acid.

31. The method of claim 22 including further drying the solution that has been applied onto the glove after the glove is no longer on the mold, and wherein
the method is performed on an automatic production line,
said glove on said mold is considered to be inside out, whereby an outwardly facing surface of said glove that is on said mold is the surface that will face a hand when said glove is worn,
said liquid carrier is water,
the applying of said coating comprises spraying or dripping said liquid carrier onto said glove while said glove is on said mold, and
the at least partially drying includes heating said glove while said glove is on said mold to remove liquid carrier from said coating that has been applied onto said glove.
